(19) United States
(12) Patent Application Publication
(10) Pub. No.: US 2026/0013822 A1
(43) Pub. Date: Jan. 15, 2026

(54) RADIOGRAPHIC IMAGING APPARATUS, METHOD, AND PROGRAM, AND PHOTON-COUNTING DETECTOR

(71) Applicant: FUJIFILM Corporation, Tokyo (JP)

(72) Inventors: Kazuma YOKOI, Tokyo (JP); Shinichi KOJIMA, Tokyo (JP)

(73) Assignee: FUJIFILM Corporation, Tokyo (JP)

(21) Appl. No.: 19/263,345

(22) Filed: Jul. 8, 2025

(30) Foreign Application Priority Data
Jul. 12, 2024 (JP) 2024-112939

(51) Int. Cl. A61B 6/58; A61B 6/00; A61B 6/42

(52) U.S. Cl. CPC A61B 6/585; A61B 6/4241; A61B 6/481

(57) ABSTRACT

Processing is performed on projection data acquired by a photon-counting detector that converts incident radiation into the number of detected photons for each of a plurality of energy bins. Material decomposition of a subject is performed based on a plurality of pieces of calibration data representing energy spectra of a predetermined calibration member and an energy spectrum of an energy region on at least one side with respect to an absorption edge of a photon energy of a material expected to be present in a body of the subject during imaging of the subject, in projection data acquired by measuring the subject with the photon-counting detector.

### CROSS REFERENCE TO RELATED APPLICATIONS

The present application claims priority from Japanese Patent Application No. 2024-112939, filed on Jul. 12, 2024, the entire disclosure of which is incorporated herein by reference.

### BACKGROUND

### Technical Field

The present disclosure relates to a radiographic imaging apparatus, method, and program, and a photon-counting detector.

### Related Art

A photon-counting computed tomography (PCCT) apparatus comprising a photon-counting detector, which is a detector employing a photon-counting method, is known. The photon-counting detector can measure a photon energy, which is an energy of photons of incident radiation. Therefore, in a PCCT apparatus, a material decomposition image in which materials with different compositions are decomposed, for example, a medical image in which an iodine contrast agent used for angiography and calcified plaques in blood vessels are decomposed by material decomposition, can be obtained. In order to obtain the material decomposition image, calibration of the detector is performed. Therefore, calibration is performed in which, for combinations of a plurality of base materials, which are materials with known compositions and thicknesses, a relationship between an output measured by the photon-counting detector and the photon energy is acquired in advance as calibration data for each detector element.

For example, JP2023-108073A and JP2021-108954A disclose a method of performing calibration using a calibration member that includes, as a first base material, a material with a relatively low effective atomic number, for example, acrylic, polyethylene, and the like, and, as a second base material, a material with a relatively high effective atomic number, for example, aluminum, a calcium mixture, an iodine mixture, tin, and the like.

Meanwhile, photon absorption (attenuation) of a material generally decreases as the photon energy increases, but it is known that there are significant points, such as a K absorption edge (also referred to as a K-edge), which exhibits a discontinuous structure of absorption at a certain photon energy. The energy position of the K-edge varies depending on the material, and the K-edge appears at a higher energy for a material with a higher atomic number.

Here, a subject may contain materials other than human body compositions, such as a drug injected into the subject as a contrast agent or artificial objects (such as gold dental fillings, bolts used to fix bones, and embolization coils for blood clots) contained in a body of the subject. Since these materials have different absorption edges for each material, it is preferable to perform calibration using these materials individually in order to perform accurate material decomposition.

However, performing calibration for all materials, including drugs expected to be used during imaging, metals expected to be contained in the body of the subject, and the like is very time and labor-consuming. In addition, the number of materials used for drugs such as contrast agents may increase in the future. For example, the use of gadolinium (atomic number 64), gold (atomic number 79), barium (atomic number 56), and the like has been considered.

### SUMMARY OF THE INVENTION

The present disclosure has been made in view of the above-described circumstances, and an object of the present disclosure is to enable effective material decomposition regardless of whether a material contained in a body of a subject has a discontinuous structure of absorption, such as a K-edge, within a measurement energy region.

According to the present disclosure, there is provided a radiographic imaging apparatus that performs processing on projection data acquired by a photon-counting detector that converts incident radiation into the number of detected photons for each of a plurality of energy bins, the radiographic imaging apparatus comprising: a storage unit that stores a plurality of pieces of calibration data representing energy spectra of a plurality of types of calibration members consisting of combinations of one or more base materials, each with at least one of a different composition or a different thickness, the plurality of pieces of calibration data being acquired by measuring the calibration members with the photon-counting detector; and a processor, in which the processor is configured to perform material decomposition of a subject based on the calibration data and an energy spectrum of an energy region on at least one side with respect to an absorption edge of a photon energy of a material expected to be present in a body of the subject during imaging of the subject, in projection data acquired by measuring the subject with the photon-counting detector.

In the radiographic imaging apparatus according to the present disclosure, the processor may be configured to perform material decomposition of the subject based on an energy spectrum on a higher-energy side with respect to the absorption edge and an energy spectrum on a lower-energy side with respect to the absorption edge in the projection data.

In the radiographic imaging apparatus according to the present disclosure, the processor may be configured to perform material decomposition of the subject based on an energy spectrum on a higher-energy side with respect to the absorption edge in the projection data and an energy spectrum of an entire energy region in the projection data.

In the radiographic imaging apparatus according to the present disclosure, the processor may be configured to perform material decomposition of the subject based on an energy spectrum on a lower-energy side with respect to the absorption edge in the projection data and an energy spectrum of an entire energy region in the projection data.

In the radiographic imaging apparatus according to the present disclosure, the processor may be configured to perform material decomposition of the subject based on an energy spectrum on a higher-energy side with respect to the absorption edge and an energy spectrum on a lower-energy side with respect to the absorption edge in the projection data, and an energy spectrum of an entire energy region in the projection data.

In the radiographic imaging apparatus according to the present disclosure, the number of the plurality of energy bins may be three or more and eight or less.

According to the present disclosure, there is provided a radiographic imaging method in a radiographic imaging apparatus that performs processing on projection data acquired by a photon-counting detector that converts incident radiation into the number of detected photons for each of a plurality of energy bins, the radiographic imaging apparatus including a storage unit that stores a plurality of pieces of calibration data representing energy spectra of a plurality of types of calibration members consisting of combinations of one or more base materials, each with at least one of a different composition or a different thickness, the plurality of pieces of calibration data being acquired by measuring the calibration members with the photon-counting detector, the radiographic imaging method comprising: performing material decomposition of a subject based on the calibration data and an energy spectrum of an energy region on at least one side with respect to an absorption edge of a photon energy of a material expected to be present in a body of the subject during imaging of the subject, in projection data acquired by measuring the subject with the photon-counting detector.

According to the present disclosure, there is provided a radiographic imaging program for causing a computer to function as a radiographic imaging apparatus that performs processing on projection data acquired by a photon-counting detector that converts incident radiation into the number of detected photons for each of a plurality of energy bins, the radiographic imaging apparatus including a storage unit that stores a plurality of pieces of calibration data representing energy spectra of a plurality of types of calibration members consisting of combinations of one or more base materials, each with at least one of a different composition or a different thickness, the plurality of pieces of calibration data being acquired by measuring the calibration members with the photon-counting detector, the radiographic imaging program causing the computer to execute: a procedure of performing material decomposition of a subject based on the calibration data and an energy spectrum of an energy region on at least one side with respect to an absorption edge of a photon energy of a material expected to be present in a body of the subject during imaging of the subject, in projection data acquired by measuring the subject with the photon-counting detector.

The technology of the present disclosure can also be applied to a program and a program product.

According to the present disclosure, it is possible to perform effective material decomposition regardless of whether a material contained in a body of a subject has a discontinuous structure of absorption, such as a K-edge, within a measurement energy region.

### BRIEF DESCRIPTION OF THE DRAWINGS

FIG. 1 is a schematic configuration diagram of a medical image capturing system comprising a radiographic imaging apparatus according to an embodiment of the present disclosure.

FIG. 2 is a graph illustrating an example of energy bin settings.

FIG. 3 is a diagram showing a hardware configuration of the radiographic imaging apparatus according to the present embodiment.

FIG. 4 is a diagram showing a functional configuration of the radiographic imaging apparatus according to the present embodiment.

FIG. 5 is a diagram illustrating a calibration method of a detector.

FIG. 6 is a graph showing a K-edge of gadolinium.

FIG. 7 is a graph illustrating material decomposition according to the present embodiment.

FIG. 8 is a flowchart showing processing performed in the present embodiment.

FIG. 9 is a graph showing another example of a method of dividing energy regions.

FIG. 10 is a graph showing still another example of the method of dividing energy regions.

FIG. 11 is a graph showing still another example of the method of dividing energy regions.

FIG. 12 is a graph illustrating another example of the energy bin settings.

### DETAILED DESCRIPTION

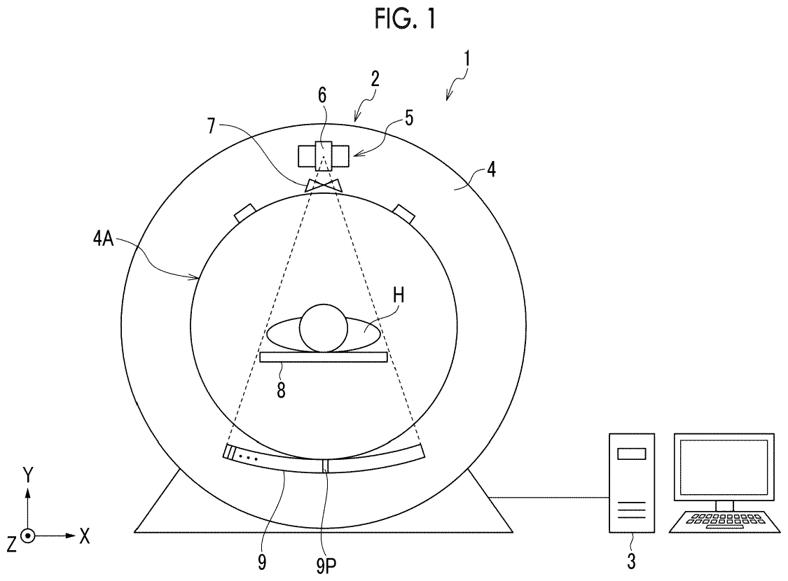

Embodiments of the present disclosure will be described in detail below with reference to the drawings. First, an example of a configuration of a medical image capturing system comprising a radiographic imaging apparatus of the present embodiment will be described. FIG. 1 is a schematic configuration diagram of the medical image capturing system comprising the radiographic imaging apparatus of the present embodiment.

As shown in FIG. 1, a medical image capturing system 1 of the present embodiment comprises a CT apparatus 2 and a console 3. The CT apparatus 2 comprises a gantry 4 and a patient table 8. In the following description, a horizontal direction in FIG. 1 is defined as an X axis, a vertical direction is defined as a Y axis, and a direction orthogonal to an XY plane is defined as a Z axis.

The gantry 4 includes an opening portion 4A, and a subject H to be imaged is disposed in the opening portion 4A in a state of being placed on the patient table 8. The gantry 4 and the patient table 8 are movable relative to each other in a Z-axis direction.

Inside the gantry 4, a radiation source 5 including a radiation tube 6 and a bowtie filter 7 and a detector 9 are disposed to face each other with the subject H interposed therebetween. The bowtie filter 7 optimizes an exposure dose by increasing the dose near the center and reducing the dose around the periphery in order to reduce the exposure dose in a peripheral portion. Radiation emitted from the radiation tube 6 is shaped into a beam shape suitable for a size of the subject H by the bowtie filter 7 and is emitted to the subject H. The detector 9 detects the radiation, which has been transmitted through the subject H, and generates projection data corresponding to the photon count of the detected radiation. As an example, the detector 9 of the present embodiment is a photon-counting detector in which a plurality of detection elements 9P, which detect a photon energy that is an energy of photons of incident radiation, are disposed in an arc shape centered on a focal point of the radiation tube 6.

In the present embodiment, X-rays are used as an example of the radiation, but the present disclosure is not limited to this, and y-rays or the like may be used.

In the present embodiment, the detector 9 detects the photon energy of incident radiation by dividing the photon energy into a plurality of energy bins. In particular, in the present embodiment, a boundary of one of the plurality of energy bins of the detector 9 is set to coincide with an absorption edge of the photon energy of a material expected to be present in a body of the subject H during imaging of the subject H.

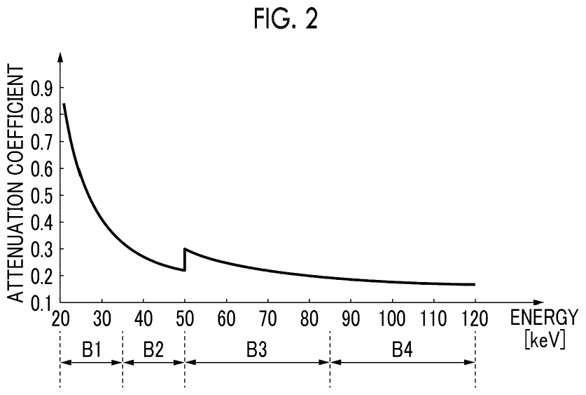

FIG. 2 is a graph illustrating an example of energy bin settings. In FIG. 2, a relationship between a radiation energy and an attenuation coefficient for a certain material expected to be present in the body of the subject H is shown. In FIG. 2, the horizontal axis represents the radiation energy (keV), and the vertical axis represents the attenuation coefficient.

Examples of the material expected to be present in the body of the subject H include iodine, gadolinium, barium, and gold used as contrast agents, embolization coils (platinum and tungsten), gold dental fillings, and bolts (titanium) for fixing bones. FIG. 2 shows the attenuation coefficient of gadolinium used as, for example, a contrast agent, and a K absorption edge (also referred to as a K-edge) showing a sudden change structure of the attenuation coefficient is observed near 50 keV.

In the present embodiment, a case where a gadolinium contrast agent is present in the subject is considered. In this case, the energy bins are set such that the boundary of one of the plurality of energy bins coincides with the K-edge of gadolinium. For example, in FIG. 2, energy bins B1 and B2 are respectively set to 20 to 35 keV and 35 to 50 keV, both of which are on a lower-energy side with respect to the K-edge, and energy bins B3 and B4 are respectively set to 50 to 85 keV and 85 keV to 120 keV, both of which are on a higher-energy side with respect to the K-edge. Consequently, a total of four energy bins are set. In the present embodiment, coinciding with the K-edge may include not only a case where the boundary completely coincides with the energy of the K-edge but also a deviation of, for example, about ±1 keV. Additionally, the energy of the K-edge may be included in the energy bin B1 or may be included in the energy bin B2.

For the energy bin B3 of 50 to 85 keV, the lower limit is 50 keV, which is the K-edge.

The radiation tube 6 and the detector 9 are rotated around the subject H by a rotation driving unit (not shown) of the gantry 4. The radiation irradiation from the radiation tube 6 and the detection of radiation by the detector 9 are repeated together with the rotation of the radiation tube 6 and the detector 9, and data (hereinafter, projection data) for the subject H is acquired for each projection path of the radiation. The projection data acquired by the detector 9 is output to the console 3 and is stored in a storage of the console 3. A value of the data corresponding to each detection element 9P, which is a minimum unit of the projection data, is the count number of photons detected by the detection element 9P. The projection data is individually acquired for each energy bin.

The dose of radiation emitted from the radiation tube 6, a rotation speed of the gantry 4, a relative movement speed between the gantry 4 and the patient table 8, and the like are set by the console 3 based on acquisition conditions for acquiring projection data, which are input from a user, such as a technician.

The console 3 of the present embodiment performs control related to acquisition of projection data, generation of a medical image, control related to material decomposition, and the like. The console 3 is an example of a radiographic imaging apparatus of the present disclosure.

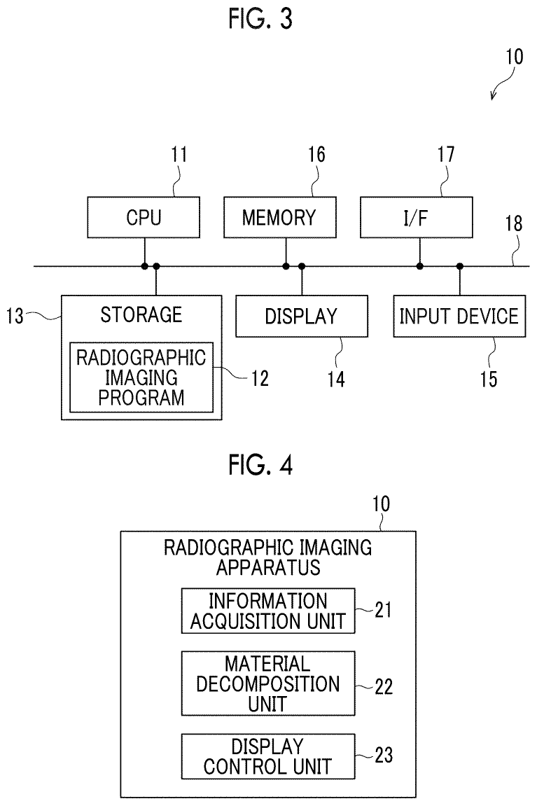

Next, the radiographic imaging apparatus according to the present embodiment will be described. First, a hardware configuration of the radiographic imaging apparatus according to the present embodiment will be described with reference to FIG. 3. As shown in FIG. 3, a radiographic imaging apparatus 10 is a computer, such as a workstation, a server computer, and a personal computer, and comprises a central processing unit (CPU) 11, a non-volatile storage 13, and a memory 16 as a temporary storage area.

In addition, the radiographic imaging apparatus 10 comprises a display 14, an input device 15, and an interface (I/F) 17. The CPU 11, the storage 13, the display 14, the input device 15, the memory 16, and the I/F 17 are connected to a bus 18. The CPU 11 is an example of a processor in the present disclosure.

The storage 13 is implemented by a hard disk drive (HDD), a solid state drive (SSD), a flash memory, or the like. A radiographic imaging program 12 installed in the radiographic imaging apparatus 10 is stored in the storage 13 as a storage medium. The CPU 11 reads out the radiographic imaging program 12 from the storage 13, loads the radiographic imaging program 12 into the memory 16, and executes the loaded radiographic imaging program 12. Additionally, calibration data, which will be described below, is stored in the storage 13. The storage 13 is an example of a storage unit of the present disclosure.

The display 14 is a device that displays various screens and is, for example, a liquid crystal display or an electro luminescence (EL) display.

The input device 15 is used by the user to input scan conditions for acquiring the projection data, instructions or various kinds of information related to generation and display of images, and the like. Examples of the input device 15 include various switches, buttons, a touch panel, a touch pen, a keyboard, and a mouse. The display 14 and the input device 15 may be integrated into a touch panel display.

The I/F 17 performs communication of various kinds of information with the rotation driving unit (not shown) of the gantry 4, the radiation source 5, and the detector 9 through wired communication or wireless communication.

The radiographic imaging program 12 is stored in a storage device of a server computer connected to a network or in a network storage in a state of being accessible from the outside and is downloaded and installed on the computer that constitutes the radiographic imaging apparatus 10 in response to the request. Alternatively, the radiographic imaging program 12 is distributed by being recorded on a recording medium, such as a digital versatile disc (DVD) or a compact disc read only memory (CD-ROM), and is installed on the computer that constitutes the radiographic imaging apparatus 10 from the recording medium.

Next, a functional configuration of the radiographic imaging apparatus according to the present embodiment will be described. FIG. 4 is a diagram showing the functional configuration of the radiographic imaging apparatus according to the present embodiment. As shown in FIG. 4, the radiographic imaging apparatus 10 comprises an information acquisition unit 21, a material decomposition unit 22, and a display control unit 23. The CPU 11 executes the radiographic imaging program 12 to function as the information acquisition unit 21, the material decomposition unit 22, and the display control unit 23.

The information acquisition unit 21 receives projection data P0 and calibration data C0 from the CT apparatus 2 via the I/F 17. The calibration data C0 is acquired by performing calibration of the detector 9 using a calibration member. Hereinafter, the calibration will be described.

In the medical image capturing system 1 comprising the detector 9 which is a photon-counting detector, the projection data for each energy bin of the subject H (that is, the energy spectrum for each projection path) can be acquired. Therefore, it is possible to generate a material decomposition image in which materials with different compositions are decomposed and a medical image divided into a plurality of energy components. In order to obtain the material decomposition image and the like in this way, it is necessary to acquire the calibration data C0 representing a relationship between an output measured by the detector 9 for combinations of a plurality of base materials, which are materials with known compositions and thicknesses, and the photon energy. The calibration refers to acquiring such calibration data C0.

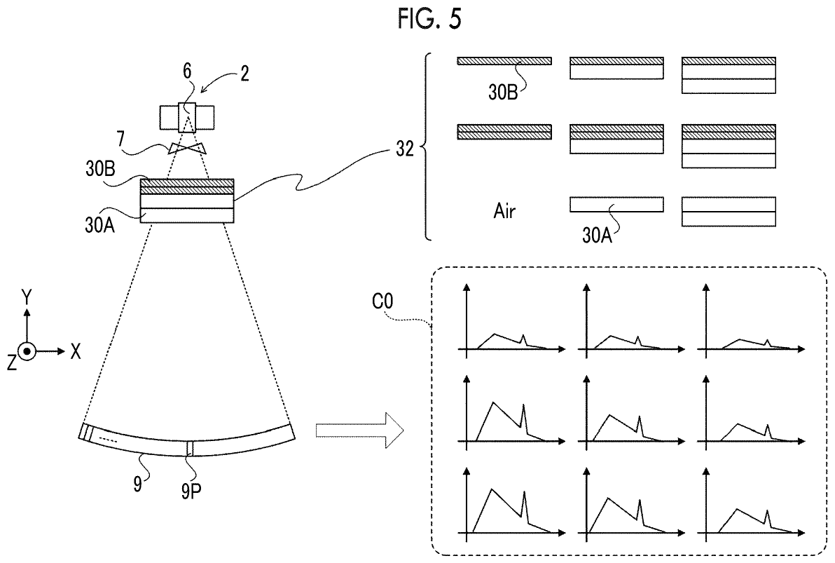

Hereinafter, an example of a calibration method of the detector 9, which is a photon-counting detector, will be described. FIG. 5 is a diagram illustrating the calibration method of the detector. A calibration member consisting of combinations of one or more base materials with known compositions and thicknesses is used for the calibration of the detector 9, which is a photon-counting detector. In FIG. 5, a calibration member 30 consists of combinations of two types of base materials, that is, a first base material 30A and a second base material 30B. The first base material 30A and the second base material 30B have different attenuation coefficients for radiation. In the present embodiment, the second base material 30B has a larger attenuation coefficient than that of the first base material 30A. Examples of the first base material 30A include acrylic (soft tissue equivalent material), and examples of the second base material 30B include aluminum (bone equivalent material) having a larger attenuation coefficient than that of acrylic.

In the example shown in FIG. 5, two sheets of unit thickness of the first base material 30A and two sheets of unit thickness of the second base material 30B are combined. In this way, the calibration data C0 is obtained for each of combinations 32 of the thicknesses of the first base material 30A and the second base material 30B in a transmission direction of the radiation. For example, in a case where the thickness of the first base material 30A is M types and the thickness of the second base material 30B is N types, M×N pieces of calibration data C0 are obtained by M×N types of combinations 32 of base materials.

Specifically, in the example shown in FIG. 5, in a case where the calibration member 30 that uses no first base material 30A is considered as a calibration member 30 with a thickness of “zero” for the first base material 30A, there are M=three types of thicknesses for the first base material 30A. Similarly, in a case where the calibration member 30 that uses no second base material 30B is considered as a calibration member 30 with a thickness of “zero” for the second base material 30B, there are N=three types of thicknesses for the second base material 30B. Therefore, in this case, there are 3×3=9 types of calibration members obtained by combining the base materials. In FIG. 5, “Air” corresponds to the calibration member 30 that uses neither the first base material 30A nor the second base material 30B, that is, the calibration member 30 with the thickness of each of the first base material 30A and the second base material 30B being “zero”.

In the present embodiment, for each of the nine types of combinations 32, the radiation source 5 emits radiation, and the detector 9 detects the radiation that has been transmitted through the combinations 32, thereby acquiring a photon energy spectrum (that is, a relationship between the radiation energy and the count number of photons) for each of the combinations 32 as the calibration data C0. The nine types of calibration data C0 acquired in this manner are output to the console 3.

In the console 3, the calibration data C0 acquired from the CT apparatus 2 is stored in the storage 13 in association with the types of the combinations 32 used to acquire the calibration data C0. The stored calibration data C0 is used for the material decomposition using the projection data P0 of the subject H.

In the present embodiment, four energy bins B to B4 are set in the detector 9. Therefore, for each of the nine types of combinations 32 of the above-mentioned base materials 30A and 30B, the calibration data C0 may be stored as a map representing the count number of photons for each of the four energy bins B1 to B4. In addition, the calibration data C0 may be represented by a graph, a mathematical formula, or the like and stored in the storage 13.

The material decomposition unit 22 derives a material decomposition image by performing material decomposition using the projection data P0 acquired by the CT apparatus 2 through imaging of the subject H. Hereinafter, the material decomposition will be described.

The projection data P0 is acquired at various projection angles in the CT apparatus 2 and has an energy spectrum of radiation for each detection element 9P provided in the detector 9. The material decomposition unit 22 searches the energy spectra represented by the calibration data C0 for an energy spectrum having a shape closest to the energy spectrum of each detection element 9P in each piece of projection data P0 and acquires a combination of the thicknesses of the base materials corresponding to the found energy spectrum. The estimation of the thickness of the base material by such a method corresponds to fitting between the energy spectrum represented by the calibration data C0 and the energy spectrum of each detection element 9P. Projection data for each base material is generated from the combination of the thicknesses of the base materials.

Here, in a case where the first base material 30A used in acquiring the calibration data C0 is acrylic and the second base material 30B is aluminum, the thickness of the acrylic and the thickness of the aluminum are obtained for each detection element 9P in the projection data P0 at various projection angles. Additionally, the projection data of acrylic and the projection data of aluminum are obtained. Then, the material decomposition unit 22 derives the material decomposition image by reconstructing a tomographic image for each base material from a plurality of pieces of projection data obtained for each base material.

In the present embodiment, as shown in FIG. 2, the detector 9 detects the photon energy of the incident radiation divided into four energy bins B1 to B4. In the present embodiment, upon the material decomposition using the projection data P0, material decomposition is performed by dividing an energy band of the projection data P0 into a higher-energy region (that is, the energy bins B3 and B4) that is higher than the K-edge of the material expected to be present in the body of the subject H and a lower-energy region (that is, the energy bins B1 and B2) that is lower than the K-edge.

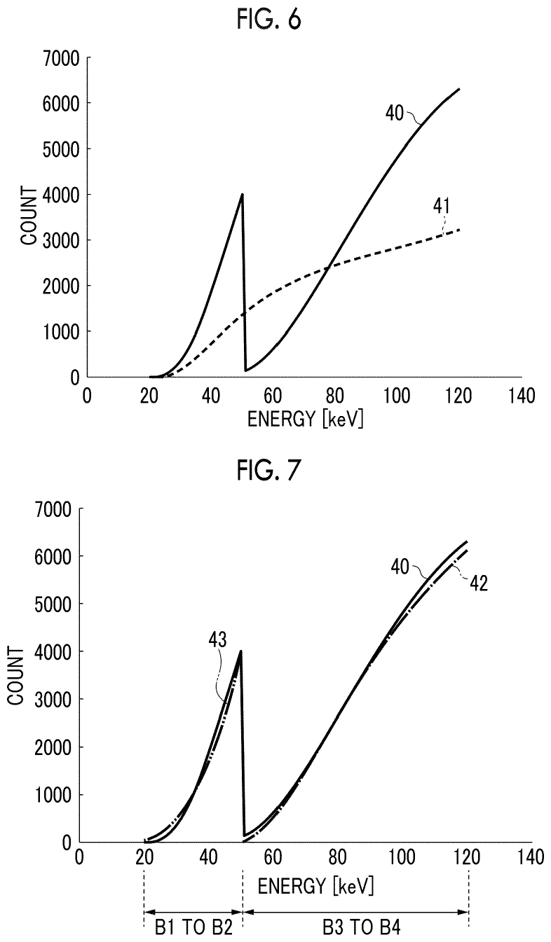

In the present embodiment, as the material expected to be present in the body of the subject H, for example, a contrast agent using gadolinium as a raw material is used. FIG. 6 is a graph showing the energy spectrum of gadolinium. In the energy spectrum shown in FIG. 6, the horizontal axis represents the radiation energy, and the vertical axis represents the count number of photons. In a case where the count number of the air is denoted as C0, it can be calculated using C0 exp(−attenuation). In FIG. 6, for the sake of clarity in the description, C0 is set to a uniform value (10000) for each energy bin. Therefore, the attenuation shown in FIG. 2 corresponds to the energy spectrum shown in FIG. 6, and the K-edge is observed near 50 keV in both cases.

As shown in FIG. 6, in a case where the K-edge is observed in an energy spectrum 40 of the projection data P0, performing material decomposition using the calibration data C0 in the entire energy region of the projection data P0 cannot perform effective material decomposition for the K-edge, as indicated by a dashed line 41. As a result, in the entire energy region of the projection data P0, a material decomposition result, which represents an intermediate value between the energy spectrum equal to or higher than the K-edge and the energy spectrum equal to or lower than the K-edge, is obtained. In this way, the material decomposition result through calibration using a base material whose K-edge structure does not coincide with that of the subject H cannot correctly represent the actual element composition. In addition, although it is ideal to use each element that may be present in the subject H as a base for calibration, this poses a problem in terms of significant labor and time costs.

In the present embodiment, the material decomposition unit 22 performs material decomposition using the energy spectrum of the projection data P0 and the calibration data C0, in the higher-energy region than the K-edge of the material expected to be present in the body of the subject H and the lower-energy region than the K-edge, as mentioned above. Consequently, as shown in FIG. 7, in the higher-energy region than the K-edge in the projection data P0, effective material decomposition can be performed using the calibration data C0 similar to the projection data P0 as indicated by an alternating long-dash and short-dash line 42. Additionally, in the lower-energy region than the K-edge in the projection data P0, as indicated by an alternating long-dash and two short-dash line 43, effective material decomposition can be performed using the calibration data C0 similar to the projection data P0. Therefore, even in a case where the material expected to be present in the body of the subject H has the absorption edge such as the K-edge, it is possible to perform effective material decomposition through calibration using a base material that has no remarkable K-edge structure in a photon energy region (for example, about 20 to 140 keV) of the radiation used in the CT apparatus 2.

Here, in a case where there is no remarkable K-edge structure on a subject H side, effective material decomposition can be performed without being affected by the K-edge even in the entire energy region in the projection data P0.

In the present embodiment, since four energy bins B1 to B4 are set in the detector 9, an average photon energy (that is, the count number) in each of the energy bins B1 to B4 is obtained as the energy spectrum of the projection data P0. In addition, the calibration data C0 also represents the photon energy in each of the energy bins B1 to B4. Therefore, material decomposition is performed using the photon energy in each of the energy bins B1 to B4 in the projection data P0 and the photon energy in each of the energy bins B1 to B4 in the calibration data C0.

The display control unit 23 displays the derived tomographic image on the display 14.

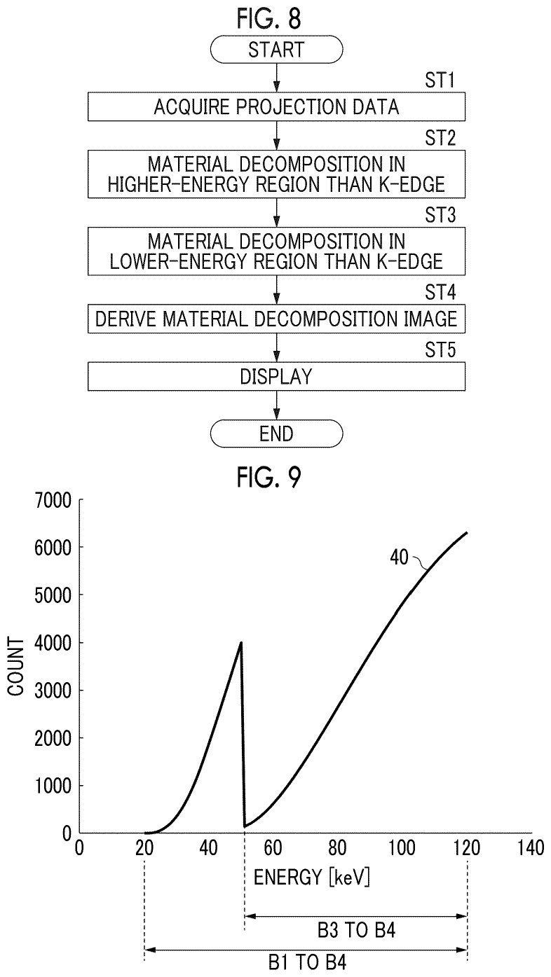

Next, processing performed in the present embodiment will be described. FIG. 8 is a flowchart showing processing performed in the present embodiment. It is assumed that the calibration data C0 is acquired in advance and stored in the storage 13. First, the information acquisition unit 21 acquires the plurality of pieces of projection data P0 derived by imaging the subject H in the CT apparatus 2 (step ST1). Next, in the higher-energy region than the K-edge of the material expected to be present in the body of the subject H, within the entire energy region that can be detected by the detector 9, the material decomposition unit 22 performs material decomposition using the energy spectrum represented by the calibration data C0 and the energy spectrum for each detection element 9P included in each of the plurality of pieces of projection data P0 (step ST2). Further, the material decomposition unit 22 performs material decomposition in the lower-energy region than the K-edge of the material expected to be present in the body of the subject H, within the entire energy region that can be detected by the detector 9 (step ST3).

Subsequently, the material decomposition unit 22 derives the material decomposition image by reconstructing the tomographic image for each base material from the plurality of pieces of projection data P0 obtained by the material decomposition for each base material (step ST4). Then, the display control unit 23 displays the material decomposition image on the display 14 (step ST5), and the processing ends.

As described above, in the present embodiment, the material decomposition of the subject H is performed based on the calibration data C0 and the energy spectrum of the energy region on at least one side with respect to the absorption edge, in the projection data P0 acquired by measuring the subject H with the detector 9. Therefore, effective material decomposition can be performed regardless of whether the material contained in the body of the subject has a discontinuous structure of absorption, such as a K-edge, within the measurement energy region.

In the above-described embodiment, material decomposition is performed by dividing the energy band of the projection data P0 into the higher-energy region (that is, the energy bins B3 and B4) that is higher than the K-edge of the material expected to be present in the body of the subject H and the lower-energy region (that is, the energy bins B1 and B2) that is lower than the K-edge of the material expected to be present in the body of the subject H, but the present disclosure is not limited to this. As shown in FIG. 9, material decomposition may be performed by dividing the energy band of the projection data P0 into the entire energy region (that is, the energy bins B1 to B4) and the higher-energy region (that is, the energy bins B3 and B4) that is higher than the K-edge of the material expected to be present in the body of the subject H.

By performing material decomposition of the subject H using the calibration data based on the energy spectrum of the entire energy region of the projection data P0 in this way, effective material decomposition of the subject H can be performed for a material having no significant point such as the K-edge. Additionally, it is possible to compare the results of the material decomposition between the entire energy region of the projection data P0 and the higher-energy region than the K-edge.

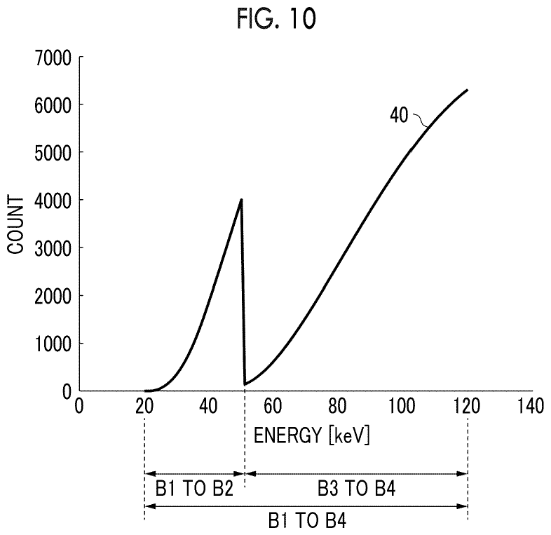

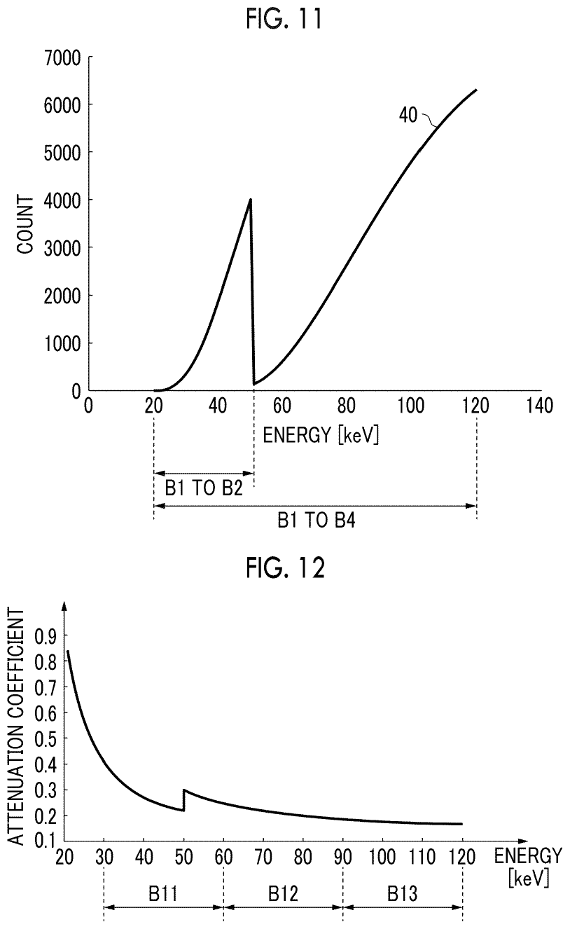

In addition, as shown in FIG. 10, material decomposition may be performed by dividing the energy band of the projection data P0 into the entire energy region (that is, the energy bins B1 to B4), the lower-energy region (that is, the energy bins B1 and B2) that is lower than the K-edge of the material expected to be present in the body of the subject H, and the higher-energy region (that is, the energy bins B3 and B4) that is higher than the K-edge. Further, as shown in FIG. 11, material decomposition may be performed by dividing the energy band of the projection data P0 into the entire energy region (that is, the energy bins B1 to B4) and the lower-energy region (that is, the energy bins B1 and B2) that is lower than the K-edge of the material expected to be present in the body of the subject H.

Additionally, in the above-described embodiment, four energy bins B1 to B4 are set for the detector 9, but the number of bins is not limited to this. Any number of two or more energy bins can be set as long as the boundary of the energy bin coincides with the K-edge of the material expected to be present in the body of the subject H. The number of energy bins is preferably three or more and eight or less because effective material decomposition cannot be performed in a case where the number of energy bins is too small, and the amount of calculation for the material decomposition increases in a case where the number of energy bins is too large.

In addition, in the above-described embodiment, the energy bin of the detector 9 is set such that the boundary of one of the plurality of energy bins coincides with the K-edge of the material, but the present disclosure is not limited to this. The boundary of the energy bin may not coincide with the K-edge. For example, as shown in FIG. 12, in a case where the energy of the K-edge is 50 keV, a total of three energy bins may be set: one energy bin B11 set at 30 to 60 keV including the K-edge and energy bins B12 and B13 respectively set at 60 to 90 keV and 90 keV to 120 keV, both of which are on the higher-energy side with respect to the K-edge.

Here, in a case where material decomposition is performed using the projection data of the energy bin B11, material decomposition need only be performed assuming that the energy band belongs to a wider side (in this case, a lower-energy side with respect to the K-edge) with respect to the energy of the K-edge within the bin. Since material decomposition into two materials requires information from two or more bins, in this case, material decomposition can be performed using two systems: the entire energy region (energy bins B11 to B13) and the energy region above the edge (energy bins B12 to B13).

Additionally, in the above-described embodiment, various processors to be described below can be used as the hardware structure of the radiographic imaging apparatus 10. The various processors include, in addition to a CPU which is a general-purpose processor that executes software (programs) to function as various processing units, a programmable logic device (PLD) whose circuit configuration can be changed after manufacturing, such as a field-programmable gate array (FPGA), a dedicated electrical circuit which is a processor having a circuit configuration dedicatedly designed for executing specific processing, such as an ASIC, and the like.

In addition, the above-described various kinds of processing may be executed using one of these various processors or may be executed using a combination of two or more processors of the same type or different types (for example, a plurality of FPGAs, a combination of a CPU and an FPGA, and the like). Alternatively, a plurality of processing units may be configured using one processor. As an example of configuring a plurality of processing units using one processor, there is an aspect in which a processor that implements all functions of a system, including a plurality of processing units, with one integrated circuit (IC) chip is used, as in a system on a chip (SOC) and the like.

Hereinafter, the supplementary claims of the present disclosure will be described.

### (Supplementary Claim 1)

A radiographic imaging apparatus that performs processing on projection data acquired by a photon-counting detector that converts incident radiation into the number of detected photons for each of a plurality of energy bins, the radiographic imaging apparatus comprising:
a storage unit that stores a plurality of pieces of calibration data representing energy spectra of a plurality of types of calibration members consisting of combinations of one or more base materials, each with at least one of a different composition or a different thickness, the plurality of pieces of calibration data being acquired by measuring the calibration members with the photon-counting detector; and
a processor,
in which the processor is configured to perform material decomposition of a subject based on the calibration data and an energy spectrum of an energy region on at least one side with respect to an absorption edge of a photon energy of a material expected to be present in a body of the subject during imaging of the subject, in projection data acquired by measuring the subject with the photon-counting detector.

### (Supplementary Claim 2)

The radiographic imaging apparatus according to Supplementary Claim 1,
in which the processor is configured to perform material decomposition of the subject based on an energy spectrum on a higher-energy side with respect to the absorption edge and an energy spectrum on a lower-energy side with respect to the absorption edge in the projection data.

### (Supplementary Claim 3)

The radiographic imaging apparatus according to Supplementary Claim 1,
in which the processor is configured to perform material decomposition of the subject based on an energy spectrum on a higher-energy side with respect to the absorption edge in the projection data and an energy spectrum of an entire energy region in the projection data.

### (Supplementary Claim 4)

The radiographic imaging apparatus according to Supplementary Claim 1,
in which the processor is configured to perform material decomposition of the subject based on an energy spectrum on a lower-energy side with respect to the absorption edge in the projection data and an energy spectrum of an entire energy region in the projection data.

### (Supplementary Claim 5)

The radiographic imaging apparatus according to Supplementary Claim 1,
in which the processor is configured to perform material decomposition of the subject based on an energy spectrum on a higher-energy side with respect to the absorption edge and an energy spectrum on a lower-energy side with respect to the absorption edge in the projection data, and an energy spectrum of an entire energy region in the projection data.

### (Supplementary Claim 6)

The radiographic imaging apparatus according to any one of Supplementary Claims 1 to 5,
in which the number of the plurality of energy bins is three or more and eight or less.

### (Supplementary Claim 7)

A radiographic imaging method in a radiographic imaging apparatus that performs processing on projection data acquired by a photon-counting detector that converts incident radiation into the number of detected photons for each of a plurality of energy bins, the radiographic imaging apparatus including a storage unit that stores a plurality of pieces of calibration data representing energy spectra of a plurality of types of calibration members consisting of combinations of one or more base materials, each with at least one of a different composition or a different thickness, the plurality of pieces of calibration data being acquired by measuring the calibration members with the photon-counting detector, the radiographic imaging method comprising:
performing material decomposition of a subject based on the calibration data and an energy spectrum of an energy region on at least one side with respect to an absorption edge of a photon energy of a material expected to be present in a body of the subject during imaging of the subject, in projection data acquired by measuring the subject with the photon-counting detector.

### (Supplementary Claim 8)

A radiographic imaging program for causing a computer to function as a radiographic imaging apparatus that performs processing on projection data acquired by a photon-counting detector that converts incident radiation into the number of detected photons for each of a plurality of energy bins, the radiographic imaging apparatus including a storage unit that stores a plurality of pieces of calibration data representing energy spectra of a plurality of types of calibration members consisting of combinations of one or more base materials, each with at least one of a different composition or a different thickness, the plurality of pieces of calibration data being acquired by measuring the calibration members with the photon-counting detector, the radiographic imaging program causing the computer to execute:
a procedure of performing material decomposition of a subject based on the calibration data and an energy spectrum of an energy region on at least one side with respect to an absorption edge of a photon energy of a material expected to be present in a body of the subject during imaging of the subject, in projection data acquired by measuring the subject with the photon-counting detector.

What is claimed is:

1. A radiographic imaging apparatus that performs processing on projection data acquired by a photon-counting detector that converts incident radiation into the number of detected photons for each of a plurality of energy bins, the radiographic imaging apparatus comprising:
a storage unit that stores a plurality of pieces of calibration data representing energy spectra of a plurality of types of calibration members consisting of combinations of one or more base materials, each with at least one of a different composition or a different thickness, the plurality of pieces of calibration data being acquired by measuring the calibration members with the photon-counting detector; and
a processor,
wherein the processor is configured to perform material decomposition of a subject based on the calibration data and an energy spectrum of an energy region on at least one side with respect to an absorption edge of a photon energy of a material expected to be present in a body of the subject during imaging of the subject, in projection data acquired by measuring the subject with the photon-counting detector.

2. The radiographic imaging apparatus according to claim 1,
wherein the processor is configured to perform material decomposition of the subject based on an energy spectrum on a higher-energy side with respect to the absorption edge and an energy spectrum on a lower-energy side with respect to the absorption edge in the projection data.

3. The radiographic imaging apparatus according to claim 1,
wherein the processor is configured to perform material decomposition of the subject based on an energy spectrum on a higher-energy side with respect to the absorption edge in the projection data and an energy spectrum of an entire energy region in the projection data.

4. The radiographic imaging apparatus according to claim 1,
wherein the processor is configured to perform material decomposition of the subject based on an energy spectrum on a lower-energy side with respect to the absorption edge in the projection data and an energy spectrum of an entire energy region in the projection data.

5. The radiographic imaging apparatus according to claim 1,
wherein the processor is configured to perform material decomposition of the subject based on an energy spectrum on a higher-energy side with respect to the absorption edge and an energy spectrum on a lower-energy side with respect to the absorption edge in the projection data, and an energy spectrum of an entire energy region in the projection data.

6. The radiographic imaging apparatus according to claim 1,
wherein the number of the plurality of energy bins is three or more and eight or less.

7. A radiographic imaging method executed by a computer in a radiographic imaging apparatus that performs processing on projection data acquired by a photon-counting detector that converts incident radiation into the number of detected photons for each of a plurality of energy bins, the radiographic imaging apparatus including a storage unit that stores a plurality of pieces of calibration data representing energy spectra of a plurality of types of calibration members consisting of combinations of one or more base materials, each with at least one of a different composition or a different thickness, the plurality of pieces of calibration data being acquired by measuring the calibration members with the photon-counting detector, the radiographic imaging method comprising:
performing material decomposition of a subject based on the calibration data and an energy spectrum of an energy region on at least one side with respect to an absorption edge of a photon energy of a material expected to be present in a body of the subject during imaging of the subject, in projection data acquired by measuring the subject with the photon-counting detector.

8. A non-transitory computer-readable storage medium that stores a radiographic imaging program for causing a computer to function as a radiographic imaging apparatus that performs processing on projection data acquired by a photon-counting detector that converts incident radiation into the number of detected photons for each of a plurality of energy bins, the radiographic imaging apparatus including a storage unit that stores a plurality of pieces of calibration data representing energy spectra of a plurality of types of calibration members consisting of combinations of one or more base materials, each with at least one of a different composition or a different thickness, the plurality of pieces of calibration data being acquired by measuring the calibration members with the photon-counting detector, the radiographic imaging program causing the computer to execute:
a procedure of performing material decomposition of a subject based on the calibration data and an energy spectrum of an energy region on at least one side with respect to an absorption edge of a photon energy of a material expected to be present in a body of the subject during imaging of the subject, in projection data acquired by measuring the subject with the photon-counting detector.
